(19) United States
(12) Patent Application Publication
(10) Pub. No.: US 2023/0210944 A1
(43) Pub. Date: Jul. 6, 2023

(54) ENTERIC-COATED GRANULAR COMPOSITION COMPRISING INGREDIENTS DERIVED FROM BEE VENOM AND LACTIC ACID BACTERIA

(71) Applicant: CHUNG JIN BIOTECH CO., LTD., Ansan-si (KR)

(72) Inventors: Jung Keun PARK, Seoul (KR); Mi Ran KANG, Ansan-si (KR); Ji Hee LIM, Ansan-si (KR); Sir A LEE, Ansan-si (KR); Joo Yeon KIM, Ansan-si (KR); Choul Koo KIM, Ansan-si (KR); Kwan Kyu PARK, Daegu (KR); Hye Min GU, Daegu (KR); Seong Jae BAE, Daegu (KR); Mi Gyeong GWON, Daegu (KR); Hyun Jin AN, Daegu (KR)

(21) Appl. No.: 17/971,277

(22) Filed: Oct. 21, 2022

(30) Foreign Application Priority Data
Dec. 30, 2021 (KR) 10-2021-0192244

(51) Int. Cl. A61K 38/17; A61K 9/50; A61K 35/745; A61K 35/747; A61K 35/744; A61P 37/04

(52) U.S. Cl. CPC A61K 38/1767; A61K 9/50; A61K 35/745; A61K 35/747; A61K 35/744; A61P 37/04

(57) ABSTRACT

Proposed is an enteric-coated granular composition containing an ingredient derived from bee venom and lactic acid bacteria. The enteric-coated granular composition containing a bee venom-derived ingredient and lactic acid bacteria according to the present invention has an immune enhancing effect by increasing cytotoxic T cells, helper T cells, or B cells and the weight of lymph nodes; and a recovery effect on kidney injury by decreasing expression of cytokine TNF-α, IL-1β and NGAL.

### BACKGROUND OF THE INVENTION

### (A) Field of the Invention

The present invention relates to an enteric-coated granular composition containing a substance derived from bee venom having an immune enhancing effect and lactic acid bacteria, and more particularly, to an immune-enhancing composition for oral administration that contains appropriate amounts of a substance obtained from bee venom by purification and lactic acid bacteria, and a manufacturing method for granulating the composition.

### (B) DESCRIPTION OF THE RELATED ART

Immunity, a self-defense system that exists in the body, is the process in which the human body recognizes, removes, and metabolizes various substances or creatures invading from the outside as foreign substances to itself. Due to immunity, the body is enabled to defend itself against damage caused by external stimuli or invasion of pathogenic microbes, but also damages to its own tissues such as causing inflammatory reactions or the like.

Bee venom is a complex mixture of enzymes, polypeptides, and other various substances with small molecular weights stored in the poison sac of a worker bee or a queen bee and is used as a defense of the bee against other animals. The known effects of the bee venom are as follows: (1) strong anti-inflammatory effect, (2) immune function control, (3) nervous system control, (4) blood circulation control, (5) biohormone secretion control, (6) antioxidant function, (7) skin whitening and wrinkle improving function, and (8) antibacterial effect. Thus, there is a need to develop more effective health functional foods using Lactobacillus in combination with the main component of the bee venom called melittin that has an immune function control effect, among other effects of the bee venom. Currently, development research related to the immune enhancing function of bee venom and application technologies using it are publically available in Korea. However, they have not been utilized for an immune-enhancing composition and a preparation method for granules for improving an immune function using melittin as an ingredient derived from bee venom and lactic acid bacteria.

### SUMMARY OF THE INVENTION

### Technical Problem

It is an object of the present invention to provide an enteric-coated granular composition containing a bee venom-derived ingredient and lactic acid bacteria with an immune enhancing effect and a recovery effect on kidney injury, and a method for preparing the same.

The above object of the present invention is not intended as a definition of the limits of the invention. The above and other objects of the invention will become apparent to those skilled in the art from the following description of embodiments.

### Technical Solution

In order to achieve the object of the present invention, there is provided an enteric-coated granular composition containing a bee venom-derived melittin and lactic acid bacteria in accordance with one aspect of the present invention.

In an embodiment, the bee venom-derived melittin may have a final melittin content of at least 60 wt. % and less than 65 wt. % with respect to melittin powder.

In an embodiment, the lactic acid bacteria may include at least one selected from the group consisting of the strains of Lactobacillus plantarum, Lactobacillus acidophilus, Lactobacillus rhamnosus, Lactobacillus casei, Lactobacillus paracasei, Lactobacillus reuteri, Lactobacillus delbrueckii ssp, Lactobacillus fermentum, Lactobacillus helveticus, Lactobacillus salivarius, Lactobacillus gasseri, Lactobacillus lactis, Lactobacillus sporogenes, Streptococcus thermophilus, Bifidobacterium longum, Bifidobacterium breve, Bifidobacterium lactis, Bifidobacterium bifium, Enterococcus faecium, Enterococcus faecalis, and the genus Leuconostoc.

In an embodiment, the bee venom-derived melittin and the lactic acid bacteria may be contained at a weight ratio of 1:10 to 1:10,000.

In an embodiment, the composition may have a granular size of 0.9 to 1.0 mm.

In an embodiment, the enteric-coated granular composition may have an immune enhancing effect by increasing at least one selected from the group consisting of cytotoxic T cells, helper T cells, and B cells, and a recovery effect on kidney injury by decreasing expression of cytokines TNF-α and IL-1β as inflammatory factors and NGAL.

In an embodiment, the enteric-coated granular composition may have an immune enhancing effect by increasing the weight of lymph nodes.

There is also provided a capsule preparation including the enteric-coated granular composition in accordance with another aspect of the present invention.

### Effects of Invention

The enteric-coated granular composition containing a bee venom-derived ingredient and lactic acid bacteria according to the present invention has an immune enhancing effect by increasing cytotoxic T cells, helper T cells, or B cells, and the weight of lymph nodes; and a recovery effect on kidney injury by decreasing expression of cytokines TNF-α and IL-1β and NGAL.

The effects of the present invention are not limited to the above effects, but should be construed to include all effects that can be inferred from the configuration of the invention disclosed in the description or claims of the present invention.

### BRIEF DESCRIPTION OF DRAWINGS

The accompanying drawings are provided a more detailed description of the present invention for those skilled in the related art and not intended to limit the scope of the present invention.

FIG. 1 is a photographic image showing the form of the enteric-coated granules (BVLS) according to a preparation example of the present invention.

FIG. 2 presents the result of comparing the solubility of the enteric-coated granules (BVLS) containing a bee venom-derived ingredient and lactic acid bacteria according to the present invention in a gastric environment at pH 3.4 and a small intestine environment at pH 6.8.

FIG. 3A and FIG. 3B presents the results of comparing the change in the body weight between the test group given 5,000 mg/kg of the enteric-coated granules (BVLS) containing a bee venom-derived ingredient and lactic acid bacteria according to the present invention and the control group to analyze the toxicity test after oral administration of BVLS, where FIG. 3A is a graph showing the change in the body weight of male rats over time; and FIG. 3B is a graph showing the change in the body weight of female rats over time.

FIG. 4 presents a graph showing the change in the weight of lymph nodes as a function of the concentration of the enteric-coated granules (BVLS) containing a bee venom-derived ingredient and lactic acid bacteria according to the present invention to analyze the BVLS immune enhancing effect test, where each point represents the mean+S.D (n=8); and no statistically significant differences were noted in the test groups from the normal control group (G1) (p<0.05, t-test).

FIG. 5A, FIG. 5B and FIG. 5C presents graphs showing the population percentages of cytotoxic T cells (FIG. 5A), helper T cells (FIG. 5B), and B cells (FIG. 5C) as a function of the concentration of the enteric-coated granules (BVLS) containing a bee venom-derived ingredient and lactic acid bacteria according to the present invention to analyze the BVLS immune enhancing effect test, where each point represents the mean+S.D (n=8); ^{¶å}p<0.01: a statistically significant difference from the normal control group (G1) by t-test; **p<0.01: a statistically significant difference from the negative control group (G2) by Dunnett t-test; and ^{##}p<0.01: a statistically significant difference from the negative control group (G2) by Steel t-test.

FIG. 6 shows the schedule of a kidney recovery effect test using the enteric-coated granules (BVLS) containing a bee venom-derived ingredient and lactic acid bacteria according to the present invention on a mouse with LPS-induced inflammation.

FIG. 7A and FIG. 7B presents the results of Western blot assays and expression level graphs showing the expression of cytokines TNF-α and IL-1β, NGAL as a marker of tubular injury, and VE-cadherin as a vascular endothelial cell marker in the blood plasma of LPS-injected mice after administration of the enteric-coated granules (BVLS) containing a bee venom-derived ingredient and lactic acid bacteria according to the present invention.

FIG. 8A and FIG. 8B presents graphs showing the creatinine and BUN levels in LPS-injected mice after administration of the enteric-coated granules (BVLS) containing a bee venom-derived ingredient and lactic acid bacteria according to the present invention in a kidney function test.

FIG. 9 presents the results of H&E staining showing the change of the kidney tissue in LPS-injected mice after administration of the enteric-coated granules (BVLS) containing a bee venom-derived ingredient and lactic acid bacteria according to the present invention.

FIG. 10 presents the results showing LPS-induced CD4 inflammation and immune response in a kidney tissue after administration of the enteric-coated granules (BVLS) containing a bee venom-derived ingredient and lactic acid bacteria according to the present invention.

FIG. 11A and FIG. 11B presents the results of IHC staining showing the changes of the kidney tissue in terms of TIM-1 and Galectin3 in LPS-injected mice after administration of the enteric-coated granules (BVLS) containing a bee venom-derived ingredient and lactic acid bacteria according to the present invention, where FIG. 11A shows a marker of tubular injury; and FIG. 11B shows a fibrosis factor in immune cells.

### DETAILED DESCRIPTION OF THE EMBODIMENTS

Hereinafter, a further detailed description will be given as to the present invention. It is provided merely for illustration of the present invention and not intended to limit the scope of the present invention. The present invention is defined only by the scope of the after-mentioned claims.

The present invention provides an enteric-coated granular composition containing a bee venom-derived melittin and lactic acid bacteria.

The bee venom is a poison made by bees and contains melittin as a main component in a content of about 40% to 50%. The main components, including melittin, consist of at least 40 substances, such as complex peptides and proteins, and active amines with a low molecular weight. Recent studies have shown that melittin contained in the bee venom can improve kidney injury and fibrosis caused after unilateral ureteral closure of mice.

In an embodiment, the bee venom-derived melittin may have a final melittin content of at least 60 wt. % and less than 65 wt. % with respect to melittin powder.

In an embodiment, the lactic acid bacteria may include at least one selected from the group consisting of the strains of Lactobacillus plantarum, Lactobacillus acidophilus, Lactobacillus rhamnosus, Lactobacillus casei, Lactobacillus paracasei, Lactobacillus reuteri, Lactobacillus delbrueckii ssp, Lactobacillus fermentum, Lactobacillus helveticus, Lactobacillus salivarius, Lactobacillus gasseri, Lactobacillus lactis, Lactobacillus sporogenes, Streptococcus thermophilus, Bifidobacterium longum, Bifidobacterium breve, Bifidobacterium lactis, Bifidobacterium bifium, Enterococcus faecium, Enterococcus faecalis, and the genus Leuconostoc. Preferably, the lactic acid bacteria may be Lactobacillus sporogenes.

In an embodiment, the bee venom-derived melittin and the lactic acid bacteria may be contained at a weight ratio of 1:10 to 1:10,000, preferably 1:100 to 1:10,000, more preferably 1:100 to 1:1,000, and most preferably 1:1,000.

In an embodiment, the enteric-coated granular composition may have a granular size of 0.9 to 1.0 mm.

An enteric-coated preparation is designed so that it can be absorbed and disintegrated only when it reaches the small intestine without disintegration in the stomach after oral administration. Therefore, this preparation should not be chewed or divided when taking it. In particular, it is the most basic and classic drug delivery technology applied when the main ingredient has a risk of being inactivated by gastric acid or causing gastrointestinal irritations.

The fluid bed process, which is one of the processes for manufacturing granules, is classified into top spray, bottom spray, and tangential spray depending on the location of the spray nozzles. Wet assembly by a fluidized bed is a step-by-step operation in an enclosed chamber and applied in many industrial fields, including pharmaceutical manufacturing, foods, and fertilizers. All the procedures of powder mixing, granulation, drying, and coating can be performed in a single container, so this method has a high yield of raw materials and shortens the process time in relation to the other granulation methods. Beside the manufacture of granules for tableting, the top spray method makes granules with a porous structure that absorbs water well and exhibits good dispersibility. Such granules can be applied to powdered foods, nutritional supplements, chemical products, etc. The fluidized bed can also be used for powder coating, powder layering, and pelletizing. The bottom spray type fluidized bed forms more excellent films compared to the other coating technologies, so it can be applied to the pharmaceutical and fertilizer industries for the purpose of adjusting or controlling the drug release. The tangential spray (rotor) method has a great application effect for pelletizing and layering.

In order to effectively prepare the enteric-coated granular composition of the present invention, freeze-dried bee venom-derived melittin powder and lactic acid bacteria powder were used.

Examples of suitable carriers, excipients or diluents available in the enteric-coated granular composition of the present invention may include at least any one selected from the group consisting of lactose, dextrose, sucrose, sorbitol, mannitol, starch, acacia gum, alginate, gelatin, oligosaccharide, dietary fiber, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil. Also, the granules of the present invention may further include a filler, an anti-agglomeration agent, a lubricant, a wetting agent, a fragrance, an emulsifier, a preservative, or the like.

The enteric-coated granular composition of the present invention may be orally administered to mammals, including rats, mice, livestock, and humans.

The dosage of the active ingredient contained in the enteric-coated granular composition of the present invention varies depending on the patient's health condition and body weight, the severity of the disease, the type of the active ingredient, and the route and duration of administration, and can be appropriately adjusted according to the patient. For example, the active ingredient may be administered in a dose of 0.0001 to 1,000 mg/kg, preferably 0.01 to 1,000 mg/kg, once or several times a day. Besides, the pharmaceutical composition of the present invention may include the active ingredient in an amount of 0.001 to 90 wt. % with respect to the total weight of the composition.

In an embodiment, the enteric-coated granular composition may have an immune enhancing effect by increasing at least any one selected from the group consisting of cytotoxic T cells, helper T cells, and B cells; or a recovery effect on kidney injury by decreasing expression of cytokines TNF-α and IL-1β as inflammatory factors and NGAL.

Cytokines TNF-α and IL-1β as inflammatory factors and NGAL as a marker of tubular injury are increased by kidney injury. They turned out to be significantly decreased by the enteric-coated granular composition of the present invention.

The kidney injury may be an acute kidney injury, and it may be a concept including all sub-diseases that occur due to damage to the kidney as a body organ. Specifically, an example of the kidney injury may even include the urinary tract disease caused by acute renal failure or kidney injury.

In an embodiment, the enteric-coated granular composition may have an immune enhancing effect by increasing the weight of lymph nodes.

In accordance with another aspect of the present invention, there is provided a capsule preparation including the enteric-coated granular composition.

Among the capsule type preparations, hard capsules may be prepared by filling conventional hard capsules with a mixture of the enteric-coated granular composition and additives such as excipients, or granules or coated granules thereof; and soft capsules may be prepared by filling a capsule base such as gelatin with a mixture of the enteric-coated granular composition and additives such as excipients. If necessary, the soft capsules may contain a plasticizer, e.g., glycerin or sorbitol, a colorant, a preservative, and so forth.

The granular preparation may be prepared in the form of granules by an appropriate method using a mixture of the enteric-coated granular composition, an excipient, a binder, a disintegrating agent, etc. If necessary, it may further contain a fragrance and a flavor enhancer.

The definitions of the terms for the excipient, the binder, the disintegrating agent, the lubricant, the flavor enhancer, the fragrance, or the like in the present invention are those described in the documents known in the related art and include those having the same or similar functions.

Hereinafter, the present invention will be described in further detail with reference to examples and experimental examples, which are given merely for illustration and not intended to limit the scope of the present invention.

### Example 1: Preparation Method for Enteric-Coated Granules (BVLS) Containing Bee Venom-Derived Ingredient and Lactic Acid Bacteria for Immune Enhancement

A bee venom-derived melittin powder, a lactic acid bacteria powder, an enteric-coated preparation, and an excipient powder were mixed at different concentration ratios to make enteric-coated granules (BVLS, Bee Venom+Lactobacillus sporogenes) containing a bee venom-derived ingredient and lactic acid bacteria for immune enhancement. The bee venom-derived melittin powder as used herein was a powder obtained by freeze-drying bee venom-derived melittin having a final melittin content of at least 60 wt. % and less than 65 wt. %.

The lactic acid bacteria powder as used herein was a lactic acid bacteria powder commercially available and including at least one lactic acid bacterium selected from the group consisting of the strains of the genus Lactobacillus (e.g., Lactobacillus plantarum, Lactobacillus acidophilus, Lactobacillus rhamnosus, Lactobacillus casei, Lactobacillus paracasei, Lactobacillus reuteri, Lactobacillus delbrueckii ssp, Lactobacillus fermentum, Lactobacillus helveticus, Lactobacillus salivarius, Lactobacillus gasseri, Lactobacillus lactis, and Lactobacillus sporogenes), the genus Streptococcus (e.g., Streptococcus thermophiles), the genus Bifidobacterium (e.g., Bifidobacterium longum, Bifidobacterium breve, Bifidobacterium lactis, and Bifidobacterium bifium), the genus Enterococcus (e.g., Enterococcus faecium and Enterococcus faecalis), and the genus Leuconostoc. The selected lactic acid bacterium was Lactobacillus sporogenes. Each lactic acid bacteria powder contained lactic acid bacteria with a viable cell count of at least 3.8×10¹⁰ cfu/g. The fluidized bed coating machine for granulation was purchased from Chung Jin Biotech Co., Ltd. (Fluid-Bed Granulator™, manufactured by Seowon ENG Co., Ltd.) and used in an experiment for preparation of enteric-coated granules.

The ingredients and the weight ratio for the manufacture of enteric-coated granules were given as in Table 1 to prepare an immune-enhancing composition. In the selected composition, the final ratio of the bee venom-derived melittin to the lactic acid bacteria was 1:1,000.

TABLE 1

| | | | Lactic acid | Enteric-coated |
|---|---|---|---|---|
| | Sieve size | Bee | bacteria powder | preparation, |
| Preparation | (16 mesh) | venom-derived | (Lactobacillus sporogenes) | binder, fluidizer, |
| Example | 0.9~1 mm | melittin powder (g) | (g) | lubricant, etc. (%) |
| 1 | Unsuitable | 1 | 20,000 | 7.2% |
| 2 | Unsuitable | 1 | 10,000 | 7.2% |
| 3 | Unsuitable | 1 | 5,000 | 7.2% |
| 4 | Unsuitable | 1 | 4,000 | 7.2% |
| 5 | Unsuitable | 1 | 3,000 | 7.2% |
| 6 | Suitable | 1 | 1000 | 7.3% |
| 7 | Unsuitable | 1 | 500 | 7.5% |
| 8 | Unsuitable | 1 | 100 | 8.6% |

(A) 500.0 g of lactose hydrate was put in the chamber of the fluidized bed coating machine, and 1.0 g of the bee venom-derived melittin powder and 1000.0 g of the lactic acid bacteria powder were added into the chamber at a constant speed.

(B) By spraying 70 to 80 ml of purified water containing 27.5 g of hydroxypropyl cellulose as a binder, the bee venom-derived melittin powder and the lactic acid bacteria powder were coated with the lactose hydrate.

(C) For enteric coating, 190.0 to 210.0 g of hydromellose, 43.0 g of carbomer (polymer), 1.2 g of silicon dioxide, and a small amount of a lubricant were dissolved in 600 to 1,000 ml of purified water to prepare an enteric coating solution. The enteric coating solution was introduced into the fluidized bed coating machine to form an enteric coating on the lactose hydrate coated with melittin and lactic acid bacteria by spraying.

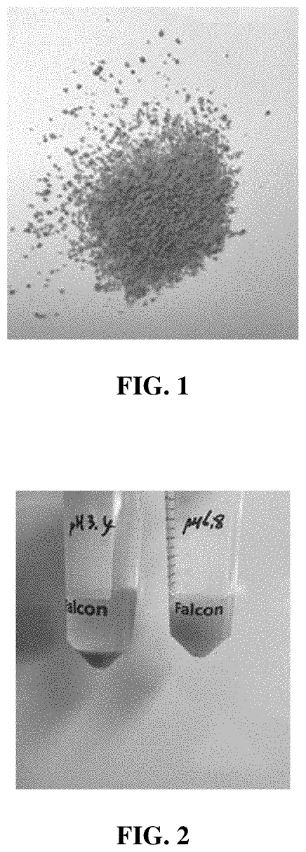

Process conditions: The total manufacturing process time was about 2 hours, including approximately 70 minutes (spraying time of 40 to 50 minutes and sorting time of about 10 minutes) at a feeding temperature of 30° C. and a drying temperature of 40 to 50° C. (average granule size: 16 mesh of 0.9 to 1.0 mm). FIG. 1 shows the form of the granules according to the preparation example of the present invention.

### [Experimental Example 1] Intestinal Solubility Test for Enteric-Coated Granules (BVLS) Containing Bee Venom-Derived Ingredient and Lactic Acid Bacteria for Immune Enhancement

In order to evaluate the intestinal solubility of the enteric-coated granules containing a bee venom-derived ingredient and lactic acid bacteria prepared according to the above-described preparation method, a gastric environment at pH 3.4 and a small intestinal environment at pH 6.8 were prepared to perform an intestinal solubility test. As a result of the experiment, the granules completely disintegrated in 4 minutes in the small intestinal environment at pH 6.8, but did not disintegrate even after 24 hours in the gastric environment at pH 3.4 (FIG. 2).

### [Experimental Example 2] Single Oral Administration Toxicity Test for Enteric-Coated Granules (BVLS) Containing Bee Venom-Derived Ingredient and Lactic Acid Bacteria for Immune Enhancement

The toxicity of the enteric-coated granules containing a bee venom-derived ingredient and lactic acid bacterial prepared according to the above-described preparation method after a single oral administration was evaluated to determine an approximate lethal dose of the granules.

A single dose was set for the subjects, which were divided into a test group (given 5,000 mg/kg of the enteric-coated granules) and a control group (given injection water). Then, a single oral administration was given to 5 male rats and 5 female rats in each group (Table 2). During the 24 days after the administration, general symptoms were observed and the body weight was measured.

TABLE 2

| | Dose | Dose volume | Number of animal subjects | |
|---|---|---|---|---|
| Group | (mg/kg) | (mL/kg) | Male | Female |
| Control | 0 | 10 | 5 | 5 |
| (G1) | | | | |
| Test group | 5,000 | 10 | 5 | 5 |
| (G2) | | | | |

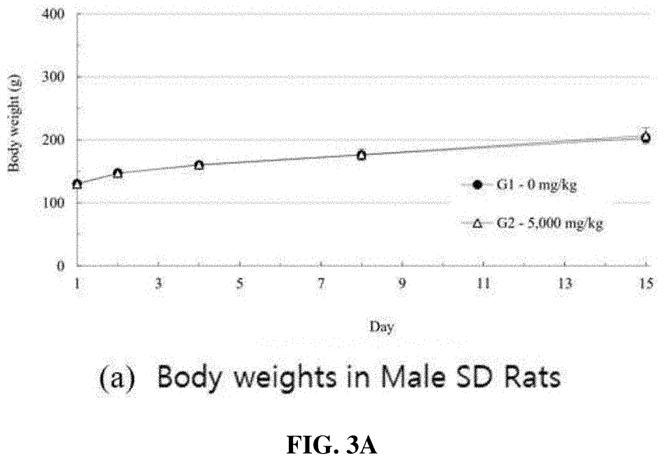

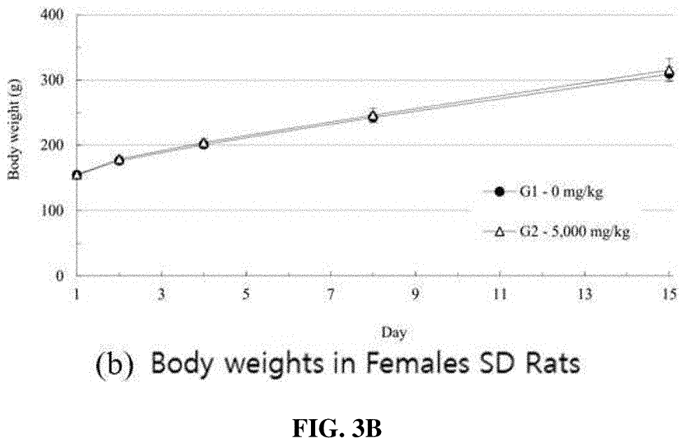

No deaths were observed in the male and female subjects in the test group (5,000 mg/kg of the enteric-coated granules). Also, neither common symptom nor any sign of effect was noted after administration of the test substance in the test group. Throughout the observation period, the male and female subjects of the test group (5,000 mg/kg) had no significant change in the body weight in comparison to those of the control group (FIG. 3A and FIG. 3B).

As a result of a single oral administration of the enteric-coated granules containing a bee venom-derived ingredient and lactic acid bacteria to rats under the conditions of this test, the approximate lethal dose was 5,000 mg/kg or above.

### [Experimental Example 3] Immune Enhancing Effect Test for Enteric-Coated Granules (BVLS) Containing Bee Venom-Derived Ingredient and Lactic Acid Bacteria for Immune Enhancement

This test was carried out to evaluate the immune enhancing effect of oral administration of the enteric-coated granules (BVLS) containing a bee venom-derived ingredient and lactic acid bacteria to male BALB/c mice immunocompromised with an immunosuppressive agent (cyclophosphamide).

The mice were divided into a normal control group (G1), a negative control group (G2), a test group (G3) given 10 mg/kg of the enteric-coated granules (BVLS), a test group (G4) given 20 mg/kg of the enteric-coated granules (BVLS), and a test group (G5) given 100 mg/kg of the enteric-coated granules (BVLS) (Table. 3).

TABLE 3

| | | | Dose | | |
|---|---|---|---|---|---|
| | | | volume | Number | Concen- |
| | Route of | Dose | (CP) | of animal | tration |
| Group | administration | (mg/kg) | (mL/kg) | subjects | (mg/mL) |
| Normal | — | — | — | 8 | — |
| control | | | | | |
| (G1) | | | | | |
| Negative | P.O. | 0 | 10 | 8 | 0 |
| control | | | | | |
| (G2) | | | | | |
| Test group | P.O. | 10 | 10 | 8 | 1 |
| 1(G3) | | | | | |
| Test group | P.O. | 20 | 10 | 8 | 2 |
| 2(G4) | | | | | |
| Test group | P.O. | 100 | 10 | 8 | 10 |
| 3(G5) | | | | | |

The body weight and immunoassay results obtained in the experiment were tested using SAS (Version 9.3, SAS Institute Inc., U.S.A.). As for the body weight and immunoassay results, Bartlett's test was used to test homogeneity of variances (significance level: 0.05). If the variances were equal across the groups, and a statistical significance was noted in the one-way analysis of variance (ANOVA)(significance level: 0.05), multiple testing of Dunnett's t-test was used to determine the statistical significance of each test group (G3, G4, G5) from the negative control group (G2) (significance level: shortened 0.05 and 0.01). When the variances were not equal across the groups, and a statistical significance was noted in the Kruskal-wallis test (significance level: 0.05), multiple testing of Steel's test was performed to determine the statistical significance of each test group (G3, G4, G5) from the negative control group (G2) (significance level: shortened 0.05 and 0.01).

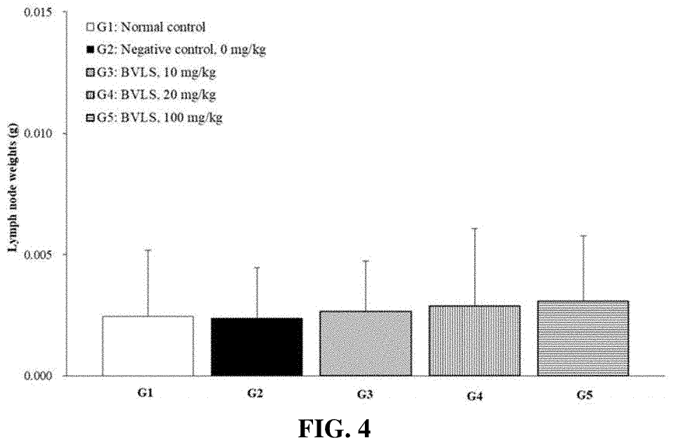

The occurrence of common symptoms was checked once a day during the observation period, and the body weight of each animal was measured twice a week. The inducing substance was administered on the 8^{th} and 11^{th} days after administration of the test substance, and blood was collected on the 1^{st}, 11^{th}, and 18^{th} days and the autopsy day to check the occurrence of immunosuppression. Serum was separated from the blood collected on the autopsy day, and lymph notes were removed. Relative to the negative control group G2, the test groups G3, G4 and G5 had an increase in the weight of the lymph nodes (FIG. 4). Such an increase in the weight of the lymph nodes in relation to that of the negative control group proved the immune enhancing effect, so this test confirmed that the test substance had an immune enhancing effect.

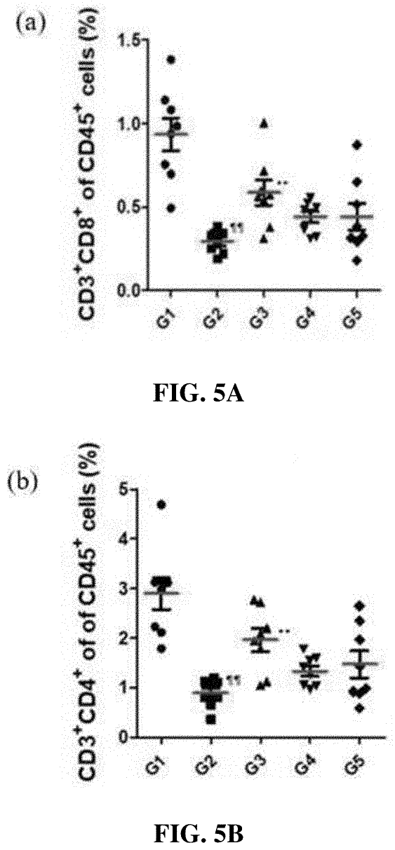

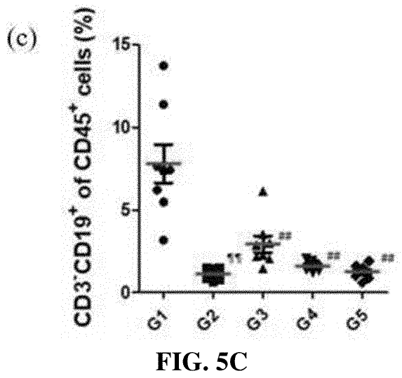

Blood was collected at the end of the observation period and subjected to an immune profile analysis. As a result, the negative control group G2 had a statistically significant decrease in the cytotoxic T cell population percentage in relation to the normal control group G1; and the test group G3 given 10 mg/kg BVLS had a statistically significant increase in the cytotoxic T cell population percentage in relation to the negative control group G2 (FIG. 5A). The negative control group G2 had a statistically significant decrease in the helper T cell population percentage in relation to the normal control group G1; and the test group G3 given 10 mg/kg BVLS had a statistically significant increase in the helper T cell population percentage in relation to the negative control group G2 (FIG. 5B). Further, the negative control group G2 had a statistically significant decrease in the B cell population percentage in relation to the normal control group G1; and the test groups G3, G4 and G5 given 10, 20 and 100 mg/kg BVLS, respectively, had a statistically significant increase in the B cell population percentage in relation to the negative control group G2 (FIG. 5C).

In conclusion, according to the immune enhancement test using male BALB/c mice immunocompromised with an immunosuppressive agent (cyclophosphamide), oral administration of a bee venom-derived substance (BVLS) as a test substance caused the test groups G3, G4 and G5 to have an increase in the weight of the lymph nodes relative to the negative control group (G2) and a statistically significant difference in the cytotoxic T cell, helper T cell and B cell population percentages from the negative control group G2, i.e., high cytotoxic T cell, helper T cell and B cell population percentages, proving the immune enhancing and improving effects of the test substance.

### [Experimental Example 4] Kidney Injury Recovery Effect Test for Enteric-Coated Granules (BVLS) Containing Bee Venom-Derived Ingredient and Lactic Acid Bacteria for Immune Enhancement

As a lipopolysaccharide (LPS) inflammation model, LPS was administered to 6-week-old female BALB/c mice through intraperitoneal injection to induce inflammation and eventually kidney injury. The mice with kidney injury were subjected to oral administration of the enteric-coated granules (BVLS) containing a bee venom-derived ingredient and lactic acid bacteria in order to evaluate the recovery effect on kidney injury.

The mice were divided into a normal control group (NC); a negative control group (LPS); a test group (NC+BVLS) given 10 mg/kg of the enteric-coated granules (BVLS) plus normal control (NC); and a test group (LPS+BVLS) given 10 mg/kg of the enteric-coated granules (BVLS) plus negative control (LPS) (Table. 4).

TABLE 4

| | Route of | Dose | Dose volume (LPS) |
|---|---|---|---|
| Group | administration | (mg/kg) | (mg/kg) |
| Normal control | — | — | — |
| (NC) | | | |
| Negative control | — | — | 10 |
| (LPS) | | | |
| Test group | P.O. | 10 | — |
| (NC + BVLS) | | | |
| Test group | P.O. | 10 | 10 |
| (LPS + BVLS) | | | |

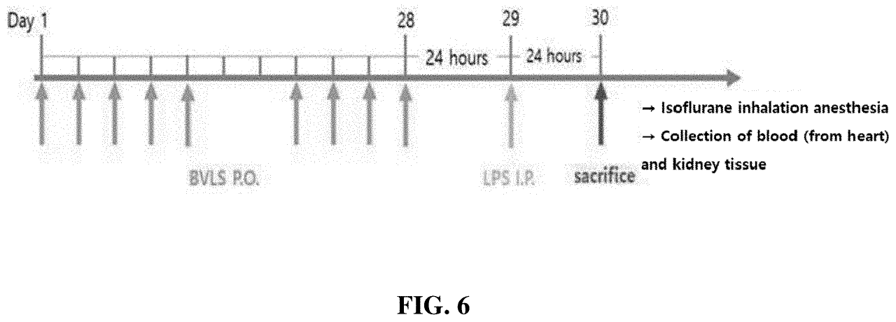

The occurrence of common symptoms was checked once a day during the observation period. The test substance was orally administered to each mouse at a concentration of 10 mg/kg five days a week for one month before LPS administration. In 24 hours after LPS injection, the blood and kidney tissues of each mouse were collected and inspected (FIG. 6).

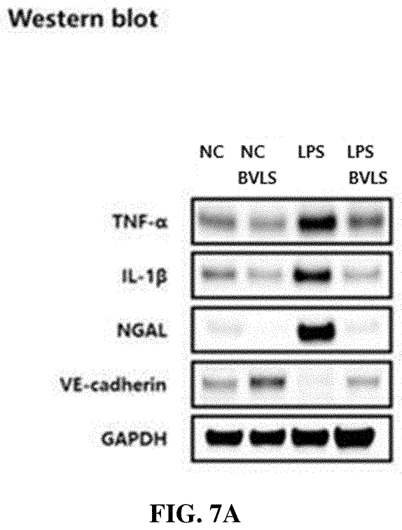

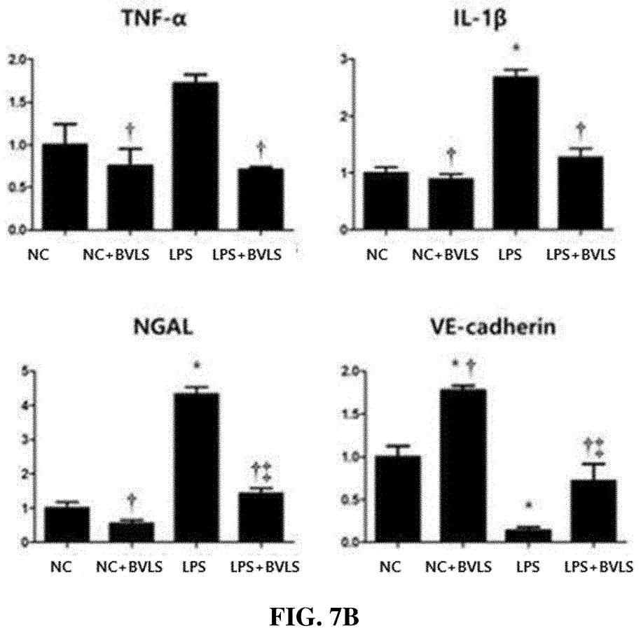

Enzyme linked immunosorbent assay (ELISA) revealed that the cytokines TNF-α and IL-1β as inflammatory factors and NGAL as a marker of tubular injury were increased by kidney injury, but significantly decreased by administration of the composition (BVLS) (FIG. 7B). Also, VE-cadherin as a vascular endothelial cell marker was increased by the injury, but increased by administration of the composition (BVLS) (FIG. 7B).

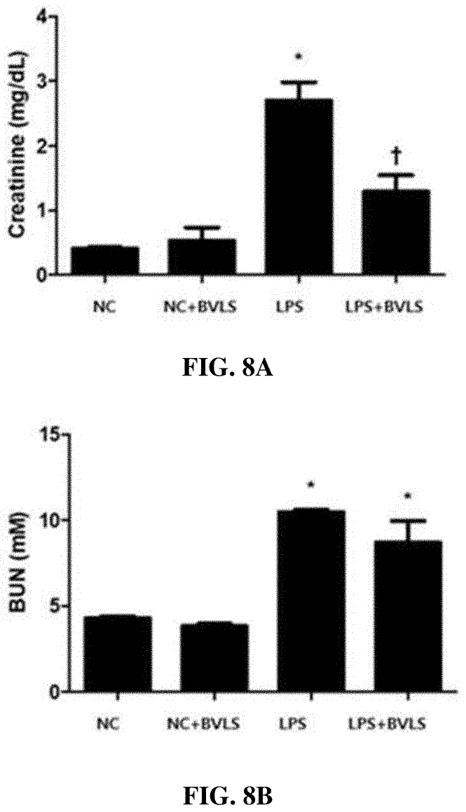

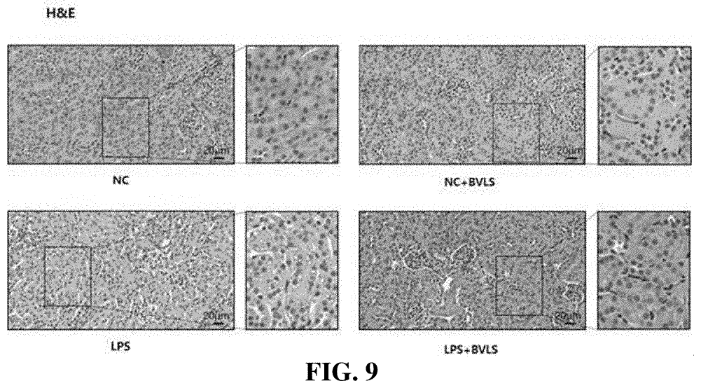

The enteric-coated granules (BVLS) containing a bee venom-derived ingredient and lactic acid bacteria exerted an effect of improving the kidney function deteriorated by LPS injection in the mice and a recovery effect on the damaged kidney tissue structure. LPS injection induced kidney dysfunction and structural damage and led to an increase in the creatinine and BUN levels in blood, indicating a severe deterioration of the kidney function, whereas BVLS administration decreased the creatinine and BUN levels in blood (FIG. 8A and FIG. 8B). H&E staining of the kidney tissue removed from the LPS-injected mice showed histopathological changes, including tubule dilatation, tubular epithelial cell damage and edema, and a significant recovery from the kidney dysfunction and structural damage after administration of the enteric-coated granules (BVLS) (FIG. 9).

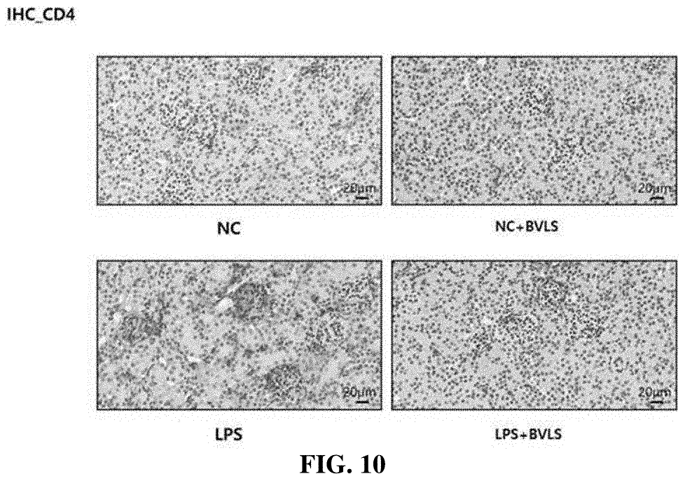

The images of the stained kidney tissue revealed the induction of inflammation and immune response in CD4 immune cells (FIG. 10).

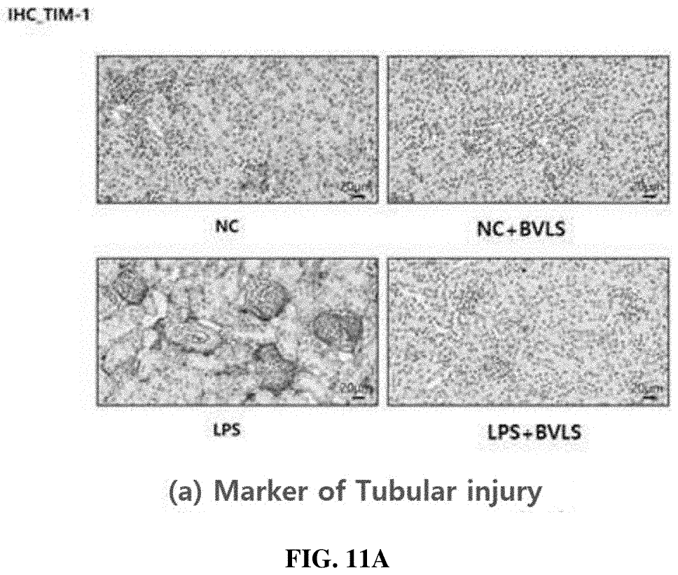

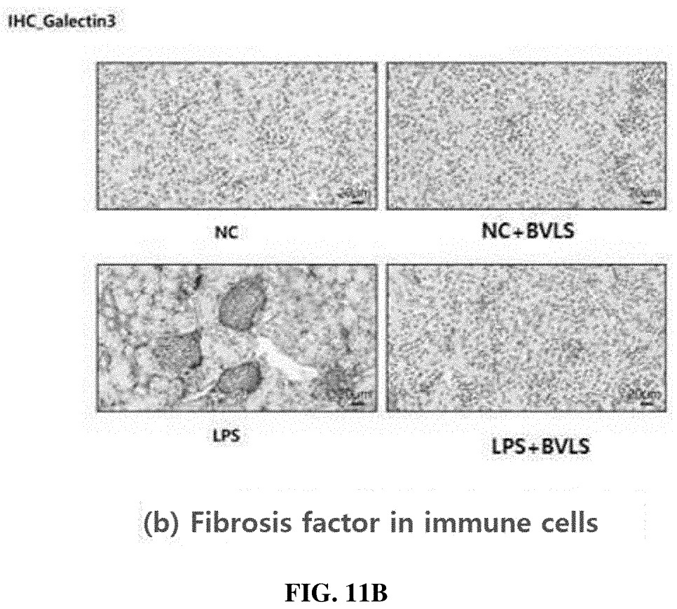

In comparison to the normal control group (NC) and the NC+BVLS test group (NC+BVLS), the negative control group (LPS) displayed a more severe tubular injury, as indicated by TIM-1 as a tubular injury marker and the part brown-colored with IHC staining (FIG. 11A). Galectin3 as a factor expressed in immune cells and playing an important role in fibrosis indicated that the negative control group (LPS) had more severe fibrosis than the normal control group (NC) or the NC+BVLS test group (NC+BVLS) (FIG. 11B).

The foregoing description of the present invention has been presented for purposes of illustration only. It should be apparent to those skilled in the present invention that many modifications and variations are possible without departing from the concept or essential features of the present invention. Therefore, the foregoing examples are to be construed as merely illustrative, and not limitative of the present invention. For example, each component described in singular form may be implemented in a scattered form; and likewise, components described as scattered may be implemented in a combined form.

The scope of the present invention is defined by the appended claims and should be construed as including all changes or modifications derived from the meaning and scope of the claims and their equivalents.

What is claimed is:

1. An enteric-coated granular composition comprising a bee venom-derived melittin and lactic acid bacteria.

2. The enteric-coated granular composition according to claim 1, wherein the bee venom-derived melittin has a final melittin content of at least 60 wt. % and less than 65 wt. % with respect to melittin powder.

3. The enteric-coated granular composition according to claim 1, wherein the lactic acid bacteria comprise at least one selected from the group consisting of the strains of Lactobacillus plantarum, Lactobacillus acidophilus, Lactobacillus rhamnosus, Lactobacillus casei, Lactobacillus paracasei, Lactobacillus reuteri, Lactobacillus delbrueckii ssp, Lactobacillus fermentum, Lactobacillus helveticus, Lactobacillus salivarius, Lactobacillus gasseri, Lactobacillus lactis, Lactobacillus sporogenes, Streptococcus thermophilus, Bifidobacterium longum, Bifidobacterium breve, Bifidobacterium lactis, Bifidobacterium bifium, Enterococcus faecium, Enterococcus faecalis, and the genus Leuconostoc.

4. The enteric-coated granular composition according to claim 1, wherein the bee venom-derived melittin and the lactic acid bacteria are contained at a weight ratio of 1:10 to 1:10,000.

5. The enteric-coated granular composition according to claim 1, wherein the composition has a granular size of 0.9 to 1.0 mm.

6. The enteric-coated granular composition according to claim 1, wherein the enteric-coated granular composition has an immune enhancing effect by increasing at least one selected from the group consisting of cytotoxic T cells, helper T cells, and B cells, and a recovery effect on kidney injury by decreasing expression of cytokines TNF-α and IL-1β as inflammatory factors and NGAL.

7. The enteric-coated granular composition according to claim 1, wherein the enteric-coated granular composition has an immune enhancing effect by increasing the weight of lymph nodes.

8. A capsule preparation comprising the enteric-coated granular composition of claim 1.
